(19) United States
(12) Patent Application Publication
(10) Pub. No.: US 2007/0118210 A1
(43) Pub. Date: May 24, 2007

(54) Trileaflet Heart Valve

(76) Inventor: Leonard Pinchuk, Miami, FL (US)

Correspondence Address: GORDON & JACOBSON, P.C., 60 LONG RIDGE ROAD, SUITE 407, STAMFORD, CT, 06902, US

(21) Appl. No.: 11/561,069

(22) Filed: Nov. 17, 2006

Related U.S. Application Data
(60) Provisional application No. 60/738,223, filed on Nov. 18, 2005.

(51) Int. Cl. A61F 2/24

(52) U.S. Cl. 623/1.26; 623/2.17

(57) ABSTRACT

A prosthetic heart valve is described that includes three leaflet members which open and close in unison with the flowing of blood through the aorta. The leaflets are made of a composite multilayer polymer material that includes a central porous material such as polyethylene terephthalate sandwiched between two other polymer layers. The two polymer layers are made up of block co-polymers that contain polyisobutylene. The composite multilayer may be formed by dip coating the porous material into a solution of the block co-polymer or by compression molding of the porous material between two layers of the block co-polymer. The composite multilayer polymer material is biocompatible and durable in bodily implant applications.

This application claims the benefit of provisional application 60/738,223, filed on Nov. 18, 2005, which is hereby incorporated by reference herein in its entirety.

### BACKGROUND OF THE INVENTION

1. FIELD OF THE INVENTION

This invention relates broadly to implantable prosthetic devices. More particularly, this invention relates to prosthetic heart valves.

2. STATE OF THE ART

Heart valve disease typically originates from rheumatic fever, endocarditis, and congenital birth defects. It is manifested in the form of valvular stenosis (defective opening) or insufficiency (defective closing). When symptoms become intolerable for normal lifestyle, the normal treatment procedure is via replacement with an artificial device or animal (e.g. pig) valve. According to the American Heart Association, in 1998 alone 89,000 valve replacement surgeries were performed in the United States (10,000 more than in 1996). In that same year, 18,520 people died directly from valve-related disease, while up to 38,000 deaths had valvular disease listed as a contributing factor.

Heart valve prostheses have been used successfully since 1960 and generally result in improvement in the longevity and symptomatology of patients with valvular heart disease. However, NIH's Working Group on Heart Valves reports that 10-year mortality rates still range from 40-55%, and that improvements in valve design are required to minimize thrombotic potential and structural degradation and to improve morbidity and mortality outcomes.

A large factor that contributes to the morbidity and mortality of patients undergoing heart valve replacement is the long length of time required on cardiopulmonary bypass as well as under general anesthesia. A heart valve that can be placed using minimally invasive techniques that reduces the amount of anesthesia and time on cardiopulmonary bypass will reduce the morbidity and mortality of the procedure.

Heart valve prostheses can be divided into three groups:

1) mechanical valves, which effect unidirectional blood flow through mechanical closure of a ball in a cage or with tilting or pivoting (caged) discs;

2) bioprosthetic valves, which are flexible trileaflet valves that are (i) aortic valves harvested from pigs, (ii) fabricated from cow pericardial tissue, and mounted on a prosthetic stent, or (iii) from cryo-preserved cadavers; and

3) polymer-based trileaflet valves.

The first group (mechanical heart valve prostheses) exhibit excellent durability, but hemolysis and thrombotic reactions are still significant disadvantages. In order to decrease the risk of thrombotic complications patients require permanent anticoagulant therapy. Thromboembolism, tissue overgrowth, red cell destruction and endothelial damage have been implicated with the fluid dynamics associated with the various prosthetic heart valves.

The second group (bioprostheses) has advantages in hemodynamic properties in that they produce the central flow characteristic to natural valves. Unfortunately, the tissue bioprostheses clinically used at present also have major disadvantages, such as relatively large pressure gradients compared to some of the mechanical valves (especially in the smaller sizes), jet-like flow through the leaflets, material fatigue and wear of valve leaflets, and calcification of valve leaflets (Chandran et al., 1989).

The use of tubular, bioprosthetic stents to support prosthetic heart valves is well known in the prior art. M. Bessler (U.S. Pat. No. 5,855,601. 1999) teaches the use of leaflet members attached to an expandable stent whereby the leaflets allow blood flow in one direction from an arterial source. S. Jayaraman (U.S. Pat. No. 6,162,245. 2000) crafts two to eight star shaped members into a chain to form an implantable stent. The stents form a central opening through which an implantable graft is received that allows vascular flow in one direction. As another example, T. Duerig (U.S. Pat. No. 6,503,272. 2003) incorporates a biocompatible fabric into an implantable stent. The biocompatible materials form the venous valve flaps. Also, G. Vardi (U.S. Pat. No. 6,835,203. 2004) uses a double implantable stent apparatus wherein a main stent serves as an anchor to a bifurcating branch stent for branching body lumens. The aforementioned patents are incorporated herein by reference in their entireties.

The third group (trileaflet valves) is desirably fabricated from biochemically inert synthetic polymers. The intent of these valves is to overcome the problem of material fatigue while maintaining the natural valve flow and functional characteristics. Clinical and commercial success of these valves has not yet been attained mainly because of material degradation and design limitations. An early attempt to form a long lasting polymeric valve incorporated thin sutures to reinforce the polymer such that the sutures acted as a series of trusses thereby preventing creep relaxation of the polymer as shown in FIG. 1. The sutures are laborious to place and are subject to variability in spacing and tautness. In addition, they are not interconnected or locked such as would be a knit or, to some extent a weave, and therefore will not demonstrate good suture retention; that is, when the leaflet is sutured to a valve frame, the reinforcing sutures will displace. Further, the reinforcing sutures must be spaced very close together to act as the load bearer—if placed too far apart, the polymer will extrude between the fibers and tear. In summary, it is difficult to place these sutures and form a functional reinforced leaflet.

### SUMMARY OF THE INVENTION

It is therefore an object of the invention to provide a trileaflet prosthetic heart valve having valve members made of a biocompatible multilayer composite polymeric material that is durable and does not cause large pressure gradients within the heart.

It is a further object of the invention to provide a prosthetic heart valve substantially made from a durable, biocompatible polymer comprised of polyisobutylene with or without block units of polystyrene.

It is yet another object of the invention to provide a prosthetic heart valve that includes a composite polymer based support structure that can be loaded with antithrombogenic or tissue growth agents.

It is still another object of the invention to provide a prosthetic heart valve which can be secured into an aortic vascular implant site by a base anchored cuff which can be affixed to a vascular wall.

It is yet another object of the invention to have a prosthetic heart valve using porous polyethylene terephthalate fabric to promote tissue growth to help symbiotically join the heart valve to the wall of a heart aorta.

It is another object of this invention to provide a method for manufacturing a prosthetic heart valve where a polymer based tubular structure is inserted through a stent and rolled up at its base to form an anchoring cuff for a vascular wall.

It is another object of this invention to provide a method for manufacturing a prosthetic heart valve where polymer based leaflets are sutured to a stent

In accord with these objects, a polymer composite material is provided that is made of a polyethylene terephthalate layer that is sandwiched between two layers of a biocompatible and biostable elastomer. This composite is biocompatible and promotes cohesive tissue interaction.

According to a first preferred embodiment, a prosthetic heart valve has leaflet members composed of a polymer composite material that allows blood flow in one direction.

According to a second embodiment, a prosthetic heart valve is depicted that has a cuff formed of an internal polymeric tubular structure where the structure is rolled up upon the base of the device to provide a means to affix the valve into an aortic vascular region.

According to another embodiment, a prosthetic heart valve is substantially made up of a polymer composite material. In this embodiment the valve is loaded with one or more antithrombogenic or therapeutic agents.

According to still another embodiment, a prosthetic heart valve is formed by positioning a porous polymer cylinder through a stent, rolling the polymer up upon itself to form a cuff, and suturing leaflet valve members to the stent so that they allow blood flow principally in only one direction.

Additional objects and advantages of the invention will become apparent to those skilled in the art upon reference to the detailed description in view of the provided figures.

### BRIEF DESCRIPTION OF THE DRAWINGS

FIG. 1 is a schematic illustration of a prior art trileaflet valve that employs sutures for polymer reinforcement.

FIG. 2A is a schematic illustration of a trileaflet valve in accordance with the present invention.

FIGS. 2B and 2C are photos of an exemplary embodiment of the trileaflet valve of FIG. 2A.

FIGS. 3A-3C are schematic diagrams of a tubular structure from which the valve leaflets and the anchoring cuff of FIG. 2A are formed.

FIG. 4 is an Scanning Electron Microscope (SEM) image of the top section of the tubular structure of FIG. 3C, which shows a composite multilayer polymeric membrane formed by dip coating in accordance with the present invention. This exemplary composite multilayer polymeric membrane can be used to form the leaflets of the valve.

FIG. 5 is an SEM image of the top section of the tubular structure of FIG. 3C, which shows a composite multilayer polymeric membrane formed by compression molding. This exemplary composite multilayer polymeric membrane can be used to form the leaflets of the valve.

FIG. 6 is a schematic illustration of the top section of the tubular structure of FIG. 3C, which shows a composite multilayer polymeric membrane preferably formed by compression molding. This exemplary composite multilayer polymeric membrane can be used to form the leaflets of the valve.

FIG. 7 is a schematic illustration of the stent element of the valve of FIG. 2A.

FIGS. 8-10 show the integration of the stent element and the composite multilayer polymeric membrane that forms the leaflets of the valve of FIG. 2A.

FIG. 11 shows the rolling up of the bottom part of the tubular structure of FIG. 2A to realize the anchoring cuff of the valve of FIG. 2A.

FIG. 12 shows the prosthetic valve implanted into the aorta of a heart secured in place by a cuff at the base of the implant

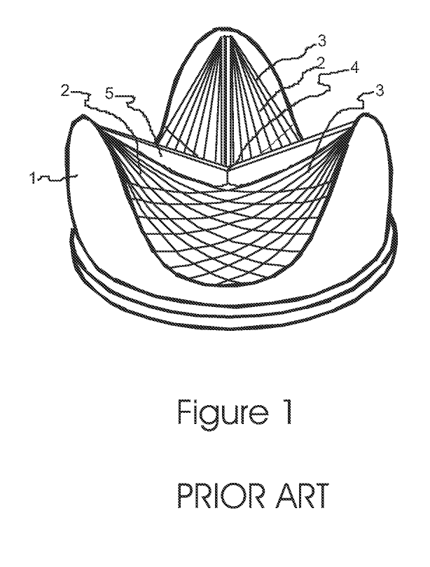

### DETAILED DESCRIPTION OF THE PREFERRED EMBODIMENTS

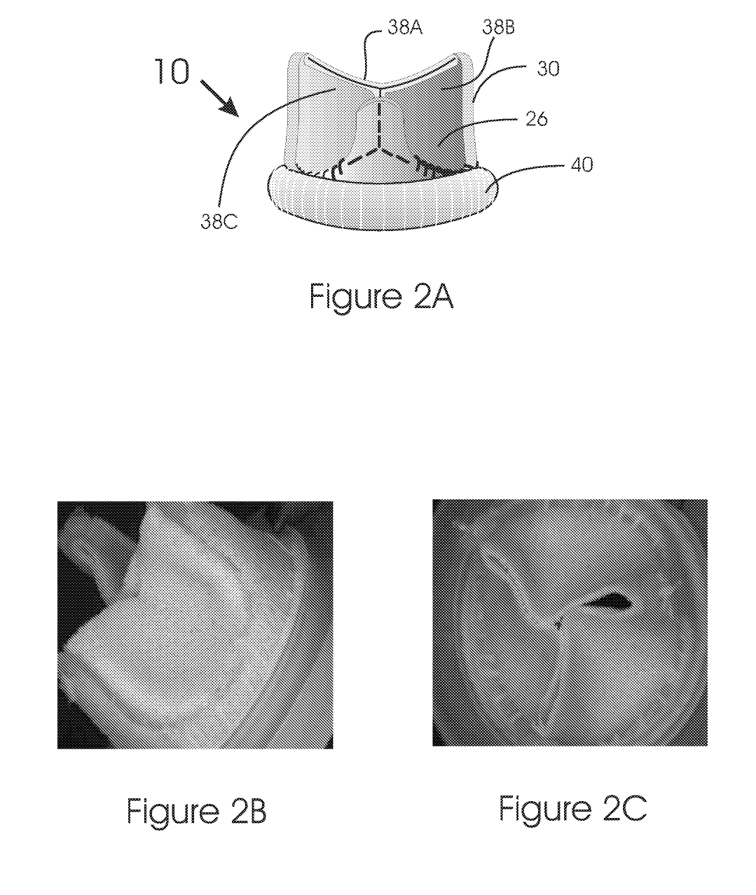

The limitations of the prior art trileaflet valves are solved by the principles of the present invention. As shown in FIG. 2A, the trileaflet valve 10 of the present invention includes a support structure shown by stent structure 30 that supports a one-piece multilayer composite polymeric membrane 26 that forms the three leaflets 38A, 38B, 38C of the valve 10. The one-piece multilayer composite polymeric membrane 26 is realized from a porous polymeric structure (e.g., a knit, weave, braid or non-woven structure) sandwiched between two outer polymer layers.

In the preferred embodiment, the central porous polymeric structure of the membrane 26 is realized from polyethylene terephthalate (PET) and the outer polymer layers of the membrane 26 are realized from a polyolefinic copolymer material containing at least one block of polyisobutylene. Other exemplary materials include crosslinked polyisobutylene, polyisobutyleneurethanes and triblock copolymers with backbones comprised of polystyrene-polyisobutylene-polystyrene, which is herein referred to as “SIBS”. SIBS can also be referred to as poly(styrene-b-isobutylene-b-styrene) where b stands for “block”. High molecular weight polyisobutylene (PIB) is a soft elastomeric material with a Shore hardness of approximately 10 A to 30 A. It is the desired anisometry of PET substrate combined with the high elasticity of SIBS coating that allows the mimicking of natural leaflet biomechanics.

When polyisobutylene is synthesized with vinyl or cyanoacrylate end groups, it can be crosslinked with heat or light and made at hardnesses up to Shore 50 A. When PIB is terminated with hydroxyl groups or amine groups and co-polymerized with polyisocyanates and chain extenders (1-4, butanediol), as are well-known in the polyurethane chemistry, the harnesses can range from Shore 60 A to Shore 100 D.

When polyisobutylene is copolymerized with polystyrene, it can be made at hardnesses ranging up to the hardness of polystyrene, which has a Shore hardness of 100 D. Thus, depending on the relative amounts of styrene and isobutylene, the SIBS material can have a range of hardnesses from as soft as Shore 10 A to as hard as Shore 100 D. In this manner, the SIBS material can be adapted to have the desired elastomeric and hardness qualities. Details of the SIBS material is set forth in U.S. Pat. Nos. 5,741,331; 6,102,939; 6,197,240; and 6,545,097, which are hereby incorporated by reference in their entireties. The SIBS material of the membrane 26 may be polymerized under control means using carbocationic polymerization techniques such as those described in U.S. Pat. Nos. 4,276,394; 4,316,973; 4,342,849; 4,910,321; 4,929,683; 4,946,899; 5,066,730; 5,122,572; and RE34,640, each herein incorporated by reference in their entireties. The styrene and isobutylene copolymer materials are preferably copolymerized in solvents. The SIBS material is preferred due to its non-inflammatory ability, its low level of encapsulation, its lack of angiogenesis, its lack of degradation, and its wide range of hardnesses as described below.

It is expected that alternative polymeric materials are suitable for the outer polymer layers of the membrane 26. Such alternative polymeric materials preferably include polyisobutylene-based material capped with a glassy segment. The glassy segment provides a hardener component for the elastomeric polyisobutylene. The glassy segment preferably does not contain any cleavable group which will release in the presence of body fluid and cause toxic side effects and cell encapsulation. The glassy segment can be a vinyl aromatic polymer (such as styrene, α-methylstyrene, or a mixture thereof), or a methacrylate polymer (such as methylmethacrylate, ethylmethacrylate, hydroxymethalcrylate, or a mixture thereof). Such materials preferably have a general block structure with a central elastomeric polyolefinic block and thermoplastic end blocks. Even more preferably, such materials have a general structure:
BAB or ABA (linear triblock),
B(AB)ₙ or a(BA)ₙ (linear alternating block), or
X-(AB)ₙ or X—(BA)ₙ (includes diblock, triblock and other radial block copolymers),
where A is an elastomeric polyolefinic block, B is a thermoplastic block, n is a positive whole number and X is a starting seed molecule.
Such materials may be star-shaped block copolymers (where n=3 or more) or multi-dendrite-shaped block copolymers. These materials collectively belong to the polymeric material referred to herein as SIBS material. Alternatively, the elastomeric material of the composite polymeric membrane 26 can be silicone rubber, polyurethane, polyolefin, copolymers of nylon, copolymers of polyester, elastin, etc. In addition, the surface of the polymer composite leaflet can be coated with surface modifying agents such as long chain hydrocarbons with silicone endgroups or fluorine end groups. In addition other agents can be adsorbed to the surface such as phospholipids and the like. Finally, drugs can be incorporated in the leaflets such as heparin, steroids, antiproliferates, and the like.

The valve 10 also includes a cuff 40 that is operably disposed on the exterior surface of the base of the stent structure 30 and used to anchor the valve 10 to the aortic annulus or similar vascular implant site. In the preferred embodiment, the cuff 40 is realized from the central porous material of the membrane 26 (e.g., PET) and formed integrally therewith as described herein. It is rolled up on itself and disposed about the exterior surface of the base of the stent structure 30. The purpose of the cuff 40 is to provide a site for suture attachment to enable fixation to the aortic annulus 42 and prevent blood from leaking around the valve 10 once in place. In addition, the porosity of the cuff 40 allows tissue ingrowth and facilitates permanent fixation of the valve 10.

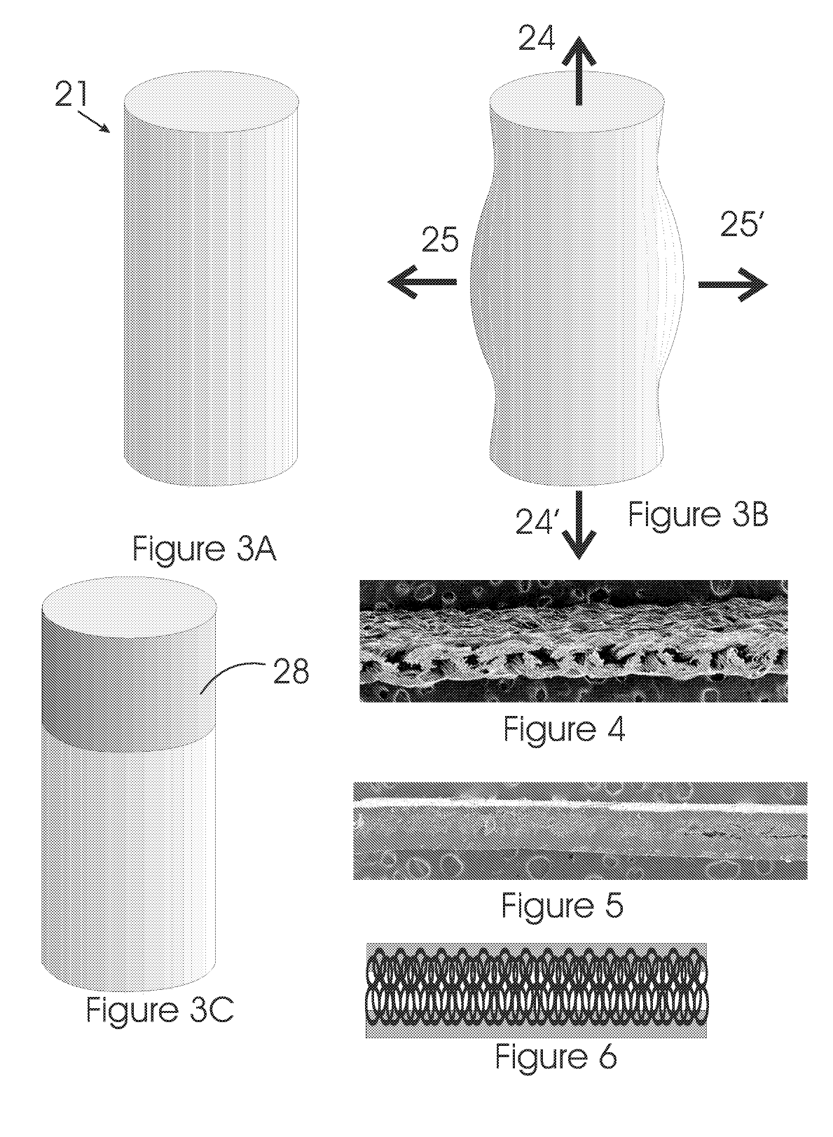

FIGS. 2B and 2C are pictures that show a side view and a top view, respectively, of an exemplary embodiment of the valve 10 of FIG. 2A. The invention is best understood by examining FIGS. 3A to 11.

FIG. 3A shows a tubular polymeric structure 21. The tubular structure 21 is porous (in other words it has air spaces or interstices therein) and can be comprised of a knit, a weave, a braid, or a non-woven structure. It is preferred that the structure 21 be a knit with some compliance in the radial or circumferential (25-25′) direction (FIG. 3B). It is also preferred that the structure be fabricated as a seamless tubular structure; however, it can also be made as a flat fabric and rolled into a tubular structure and heat welded, sutured or bonded into a tubular structure. The tubular structure while preferably substantially circular in cross-section (i.e., a cylindrical tube) may also have other cross-sectional shapes including oval and oblong. The porous tubular structure 21 is preferably realized by polymeric fibers or strands with a spacing on the order of 10 to 1,000 microns; preferably 300 microns+/−100 microns. FIG. 3B shows the tubular structure 21 with arrows 24, 24′ in the longitudinal or axial direction and arrows 25, 25′ in the radial direction. The tubular structure 21 can be stretched in the radial direction 25, 25′ but not significantly in the axial direction 24, 24′. In the preferred embodiment, the porous tubular structure 21 is realized from a polyethylene terephthalate (PET) knitted fabric, where the knit is a locked warp knit. Locking of the knit implies that it will not run if a fiber is broken which often occurs with weft or jersey knits such as that used in Nylon stockings.

As shown in FIG. 3C, a top section 28 of the porous material of the tubular structure 21 is coated on both of its sides with a polymeric material. This top section 28 will form the multilayer composite polymeric membrane 26 of the three leaflets 38A, 38B, 38C of the valve 10 as described herein.

The coating process of the top section 28 can be accomplished by dipping the top end of the cylinder structure 21 in a lacquer comprised of a polymer in a solvent and allowing the solvent to flash off. In the preferred embodiment, the coated polymer is a SIBS material as described herein. However, other polymers can be used for the coating, for example, silicone rubber, polyurethane, polybutadiene, poly(styrene-ethyelenebutylene-styrene) (SEBS), crosslinked polyisobutylene and the like. Typically any elastomeric material can be used, preferably those materials that display minimal biodegradation and good hemocompatibility. Suitable solvents include non-polar solvents such as heptane, hexane, toluene, cyclopentane, methylcyclohexane, cyclohexane, tetrahydrofuran, and the like. Solids contents preferably range from 5% to 20% with 7%-15% even more preferred. The preferred hardness of the coating of the top section 28 is between Shore 20 A and 50 A. The lower the hardness, the lower the bending moment and the higher the flex fatigue life. For SIBS material, the hardness is controlled by the mole percent styrene content. The range of styrene content is preferably in the range from 4% to 16%; more preferably in the range from 6% to 10%, and most preferably on the order of 8%.

In the event that the tubular structure 21 is dip coated, the resultant coated structure takes on the appearance of that shown in the SEM image of FIG. 4. FIG. 4 shows an edge orientation with many spaces remaining in the central porous fabric material. In addition, FIG. 4 shows a surface section of the coated cylinder with a surface that is relatively rough; that is, the cast polymer follows the topography of the central fibrous structure. When a solvent or dip cast structure dries, the polymer begins to shrink to close up the void spaces left by the evaporating solvent. Dip-coated structures of this nature have forces that tend to tighten the resultant composite structure.

Alternatively, the porous material of the top section 28 of tubular structure 21 can be coated on both of its sides with a polymeric material by compression molding. This compression molding is performed by placing a band of the polymeric material (e.g., SIBS) on a ridged mandrel concentrically within the tubular structure 21 and placing a similar band of polymeric material (e.g., SIBS) concentrically over the tubular structure 21. The assembly can be heated on a compression molding press, and with the use of a cylindrical clam shell mold, the polymeric bands can be melted and forced into the interstices of the porous tubular structure 21 (e.g., PET fabric). FIG. 5 shows an SEM image of the cross-section of such a composite structure. It can be observed that the surface of the specimen is uniform and smooth as compared to the relatively rough surface of the dip-coated structure in FIG. 4. The cross-section indicates that the central porous material (e.g., PET fabric) is penetrated by the surrounding polymeric material (e.g., SIBS).

In another alternative, the porous material of top section 28 of the structure 21 can be coated on both of its sides with a polymeric material in a manner whereby the outer polymeric material is not forced entirely through the central porous material as shown in the cross-section of FIG. 6. This configuration can readily be accomplished by compression molding with correct fixturing and control over the thickness of the multilayer sandwich. Note that the outer polymeric layers (e.g., SIBS) are pressed into respective sides of the central porous material (e.g., PET fabric) but not entirely therethrough to the extent that there is a plane of the central fabric material that is not integrated with the outer polymeric layers. This allows the outer polymer layers on either side of this central fabric material plane to slide slightly relative to each other and decreases the forces required to bend the composite material (as compared to the forces required to bend the composite structure of FIG. 5).

Note that the internal stresses within the composite structures of FIGS. 5 and 6 are outward. That is, when an elastomer is compression molded, it wants to rebound when the mold is opened and this rebound tends to loosen the composite structure. However, when dip-coated as in the cross-section of FIG. 4, the internal stresses are inward and the composite structure tends to be tight.

Also note that the composite structure formed via dip coating (FIG. 4) appears white due to the refractive index differences between the outer polymer layers and the air spaces in the central porous fabric. Similarly, the composite structure of FIG. 6 also appears white. In contrast, the composite structure of FIG. 5 where the polymer bands are forced entirely through the central porous material is relatively transparent.

Finally, note that the composite structure of FIG. 6 provides the lowest relative bending moment, the composite structure of FIG. 5 provides the next lowest relative bending moment, and the composite structure of FIG. 4 provides the highest relative bending moment. A lower bending moment provides better fatigue life for a similar structure as the stresses within the composite are less. In this manner, the composite structure of FIG. 6 provides the highest relative fatigue life, the composite structure of FIG. 5 provides the next highest relative fatigue life, and the composite structure of FIG. 4 provides the lowest relative fatigue life.

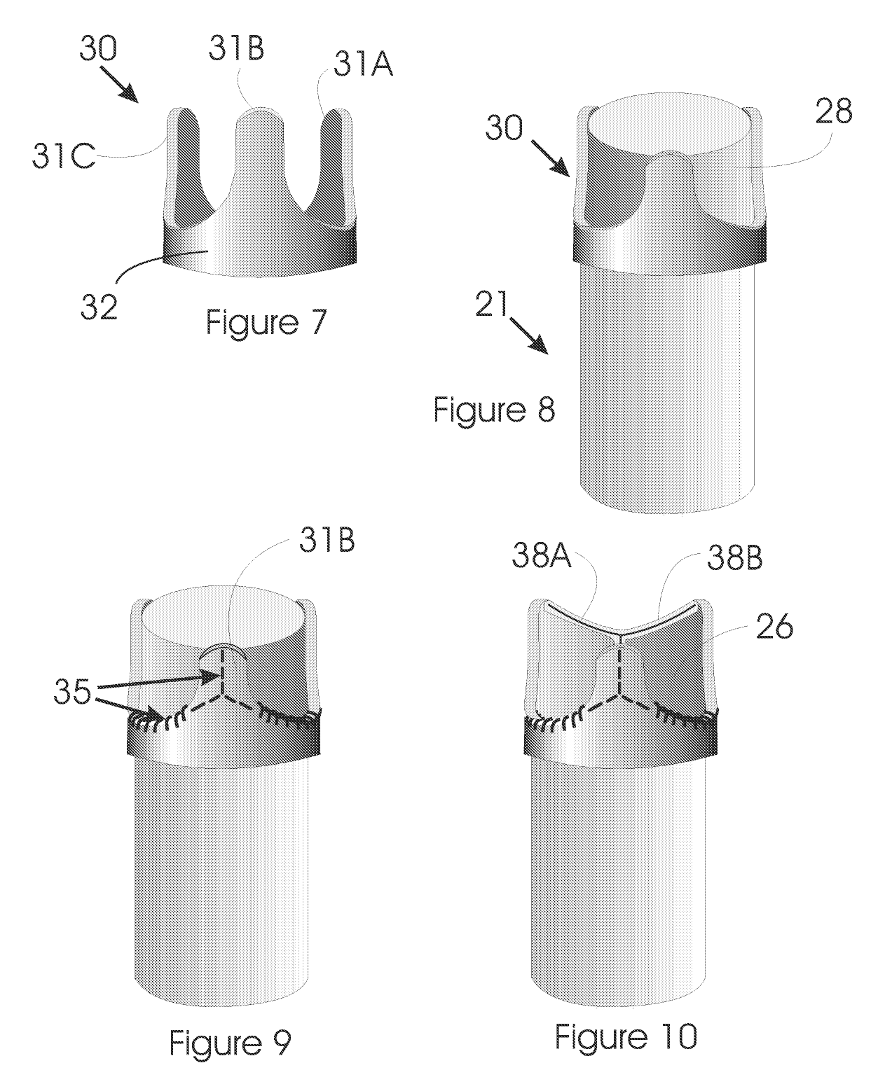

FIG. 7 shows details of the stent 30 that supports the composite polymeric membrane 26 previously described. The stent 30 includes three struts 31A, 31B, 31C that extend substantially vertical from an annular base 32. That is, the struts extend parallel to a central axis A of the tubular structure 21 (FIG. 8). The stent 30 is typically made of a more rigid material than the composite membrane 26 of the leaflets. However, the stent 30 need not be entirely rigid and allow some bending of the struts. Bending transfers some of the load energy dispersement from the leaflets to the stent 30 and helps in the longevity of the device. The stent 30 can be made from one or more polymeric materials or from one or more metals. Preferred polymers include polycarbonate, polytetrafluoroethylene, polyurethane, polysulphone, polyimid, polyamide, polyester, SIBS material, and the like. For bonding purposes, the preferred material for the stent 30 is SIBS material with a hardness of Shore 50 A to Shore 75 D; preferably Shore 75 D. These hardnesses are attained with mol percent styrene of 25% to 60%; preferably 30% to 40%; most preferred 35%. Alternatively, the stent 30 can be made from metals such as titanium, stainless steel, nitinol and the like.

FIG. 8 shows the stent 30 placed over the multilayer composite polymeric section 28 formed at the top of the tubular structure 21. A solvent such as toluene can be brushed onto the base 32 (FIG. 7) of the stent 30 to enable solvent bonding of the composite polymeric section 28 to the stent 30 in the base area only.

As shown in FIG. 9, sutures 35 can be used to secure the composite polymeric section 28 to the base 32 of the stent to reinforce the bond therebetween. Note that sutures 35 are attached to the stent 30 in the areas between the struts 31A, 31B, 31C and in a narrow seam extending up each strut as the leaflets need to be attached in this narrow seam (as opposed to the broad width of the struts as would be the case if the composite polymeric section 28 was bonded to the stent 30 in these areas).

The composite polymeric section 28 is then pinched and heat-formed into three leaflets 38A, 38B, 38C that are normally-closed as shown in FIG. 10. The method of pinching the leaflets and heat-forming them in the “normally-closed” position can be performed in many ways, such as, placing clips on the leaflets, placing a forming die over the leaflets, etc. Regardless of the method of holding them together in an opposed manner, once apposed, the structure is placed in an oven at the softening point of the polymer and the leaflets are thermoformed into the “normally-closed” position. A suitable temperature range for PET/SIBS composites is 120° C. to 170° C.; preferably 140° C.

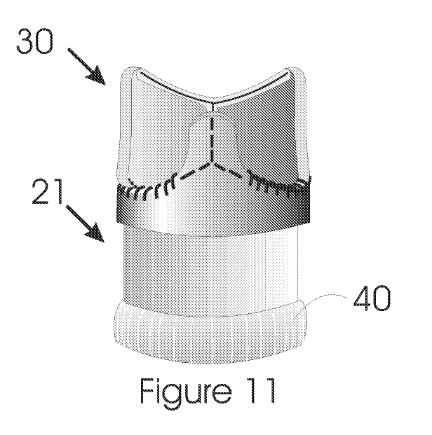

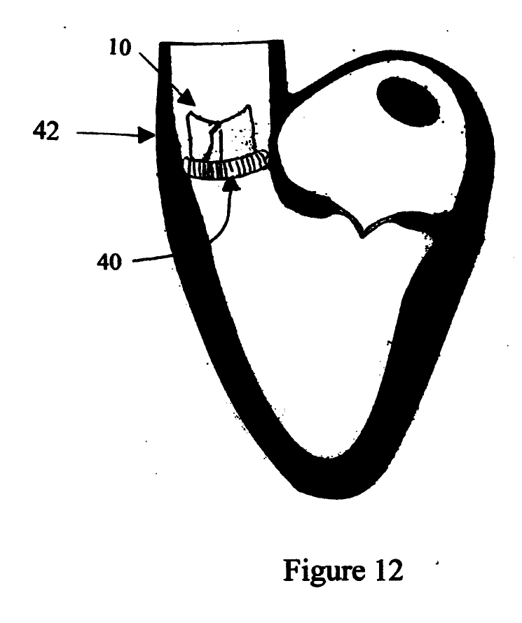

FIG. 11 shows the bottom section of the porous tubular structure 21 being rolled up to form the cuff 40 that is used anchor the valve 10 to the aortic annulus. The cuff 40 is substantially annular meaning that it may be circular, oval, or in an unevenly rolled shape such that the entirety of cuff 40 does not occupy a single plane. Once rolled up, the upped edge of cuff 40 can be sutured to the tubular structure 21 to keep it in place.

One of the tests required by the FDA prior to approving a heart valve for human testing requires that the valve be placed in a heart simulator where the valve is cycled between 90 and 110 mmHg pressure. Further, 90% of the stroke cycle requires back pressure on the leaflets of 90 mmHg. In other words, if the stroke is one second in duration; 0.1 sec is forward flow to a peak pressure of 110 mmHg through the valve and 0.9 sec is back pressure on the valve of 90 mmHg. These forces are rather severe and the FDA requires demonstration of fatigue life to 600 million cycles prior to beginning clinical studies. Importantly, the valve 10 with the leaflets realized from a multilayer composite polymeric structure as described herein have survived for 600 million cycles without failure, and thus provides longevity without creep elongation and flex fatigue wear. In addition, the design does not allow significant regurgitation (back flow) of fluid in the heart simulator. Valves of this nature have been successfully implanted in the aortic position in sheep.

The above describes a tubular structure where the tube is pinched and heat set to provide the valve in a normally closed manner. Alternatively, individual leaflets can be fabricated and attached individually to the stent with adhesives and sutures. The leaflets used in this manner are of the SIBS/PET construct described herein.

The polymer comprising the SIBS composite leaflet membrane can be coated or loaded with and released from the matrix an antithrombotic agent to prevent blood from clotting on the leaflets in vivo. Suitable antithrombotic agents include: phosphatidylcholine; preferably 2-methacryloyloxyethyl phosphorylcholine (MPC); dimyristoylphosphatidylcholine (liquid-crystalline state); Prostacyclin like 10,10-difluoro-13-dehydroprostacyclin (DF2-PGl2); double-chained, zwitterionic phospholipid 1,2-dilauroyl-sn-phosphatidylcholine (DLPC, C12); Polysaccharides like hyaluronic acid and alginic acid: heparin, heparin analogues or derivatives such as hiruden, urokinase and PPack (dextrophenylalanine proline arginine chloromethylketone).

Anti-coagulants can also be incorporated such as D-Phe-Pro-Arg chloromethyl ketone, RGD peptide-containing compounds, heparin, hirudin, antithrombin compounds, platelet receptor antagonists, anti-thrombin antibodies, anti-platelet receptor antibodies, aspirin, prostaglandin inhibitors, platelet inhibitors, and tick antiplatelet peptides.

If desired, a therapeutic agent of interest can be loaded at the same time as the polymer from which the device is realized, for example, by adding it to a polymer melt during thermoplastic processing or by adding it to a polymer solution during solvent-based processing. Alternatively, a therapeutic agent can be loaded after formation of the device or device portion. As an example of these embodiments, the therapeutic agent can be dissolved in a solvent that is compatible with both the device polymer and the therapeutic agent. Preferably, the device polymer is at most only slightly soluble in this solvent. Subsequently, the solution is contacted with the device or device portion such that the therapeutic agent is loaded (e.g., by leaching/diffusion) into the copolymer. For this purpose, the device or device portion can be immersed or dipped into the solution, the solution can be applied to the device or component, for example, by spraying, printing dip coating, immersing in a fluidized bed and so forth. The device or component can subsequently be dried, with the therapeutic agent remaining therein.

In another alternative, the therapeutic agent may be provided within a matrix comprising the polymer of the device. The therapeutic agent can also be covalently bonded, hydrogen bonded, or electrostatically bound to the polymer of the device. As specific examples, nitric oxide releasing functional groups such as S-nitroso-thiols can be provided in connection with the polymer, or the polymer can be provided with charged functional groups to attach therapeutic groups with oppositely charged functionalities.

In yet another alternative embodiment, the therapeutic agent can be precipitated onto one or more surfaces of the device or device portion. These one or more surface(s) can be subsequently covered with a coating of polymer (with or without additional therapeutic agent) as described above.

It also may be useful to coat the polymer of the device (which may or may not contain a therapeutic agent) with an additional polymer layer (which may or may not contain a therapeutic agent). This layer may serve, for example, as a boundary layer to retard diffusion of the therapeutic agent and prevent a burst phenomenon whereby much of the agent is released immediately upon exposure of the device or device portion to the implant site. The material constituting the coating, or boundary layer, may or may not be the same polymer as the loaded polymer. For example, the barrier layer may also be a polymer or small molecule from a large class of compounds.

It is also possible to form a device (or device portion) for release of therapeutic agents by adding one or more of the above or other polymers to a block copolymer. Examples include the following:
blends can be formed with homopolymers that are miscible with one of the block copolymer phases. For example, polyphenylene oxide is miscible with the styrene blocks of polystyrene-polyisobutylene-polystyrene copolymer. This should increase the strength of a molded part or coating made from polystyrene-polyisobutylene-polystyrene copolymer and polyphenylene oxide.
blends can be made with added polymers or other copolymers that are not completely miscible with the blocks of the block copolymer. The added polymer or copolymer may be advantageous, for example, in that it is compatible with another therapeutic agent, or it may alter the release rate of the therapeutic agent from the block copolymer (e.g., polystyrene-polyisobutylene-polystyrene copolymer).
blends can be made with a component such as sugar (see list above) that can be leached from the device or device portion, rendering the device or device component more porous and controlling the release rate through the porous structure.

The release rate of therapeutic agent from the therapeutic-agent-loaded polymers of the present invention can be varied in a number of ways. Examples include:
varying the molecular weight of the block copolymers;
varying the specific constituents selected for the elastomeric and thermoplastic portions of the block copolymers and the relative amounts of these constituents;
varying the type and relative amounts of solvents used in processing the block copolymers;
varying the porosity of the block copolymers;
providing a boundary layer over the block copolymer; and
blending the block copolymer with other polymers or copolymers.

Moreover, although it is seemingly desirable to provide control over the release of the therapeutic agent (e.g., as a fast release (hours) or as a slow release (weeks)), it may not be necessary to control the release of the therapeutic agent.

Hence, when it is stated herein that the polymer is “loaded” with therapeutic agent, it is meant that the therapeutic agent is associated with the polymer in a fashion like those discussed above or in a related fashion.

In addition, the suture cuff can be loaded with drugs that aid in healing or ingrowth of the suture cuff to the natural tissue of the aorta. Exemplary drugs include vascular cell growth promoters such as growth factors, transcriptional activators, and translational promoters.

Other drugs that can regulate the environment around the heart valve include protein kinase and tyrosine kinase inhibitors (e.g., tyrphostins, genistein, quinoxalines), prostacyclin analogs, cholesterol-lowering agents, angiopoietins, antimicrobial agents such as triclosan, cephalosporins, aminoglycosides and nitrofurantoin, and oligodynamic metals, cytotoxic agents, cytostatic agents, and cell proliferation affectors. In addition, combinations of the above therapeutic agents can be used.

A wide range of therapeutic agent loadings can be used in connection with the above block copolymers comprising the leaflets, with the amount of loading being readily determined by those of ordinary skill in the art and ultimately depending upon the condition to be treated, the nature of the therapeutic agent itself, the means by which the therapeutic-agent-loaded copolymer is administered to the intended subject, and so forth. The loaded copolymer will frequently comprise from less than one to 70 wt % therapeutic agent.

In some instances, therapeutic agent is released from the device or device portion to a bodily tissue or bodily fluid upon contacting the same. An extended period of release (i.e., 50% release or less over a period of 24 hours) may be preferred in some cases. In other instances, for example, in the case where enzymes, cells and other agents capable of acting on a substrate are used as a therapeutic agent, the therapeutic agent may remain within the copolymer matrix.

Advantageously, the valve device 10 as shown in FIG. 2A is readily collapsible in the radial direction such that it can be loaded into a catheter for deployment in the aorta via catheterization. In this configuration the valve stent is essentially a wire stent such as those used to stent the vasculature.

It has been described and illustrated herein a preferred embodiment of a prosthetic heart valve device (and corresponding method of production) that is positioned into the aorta of a human heart. While particular embodiments of the invention have been described, it is not intended that the invention be limited thereto, as it is intended that the invention be as broad in scope as the art will allow and that the specification be read likewise. It will therefore be appreciated by those skilled in the arts of prosthetic design and manufacture that yet other modifications could be made to the provided invention without deviating from its spirit and scope as claimed.

What is claimed is:

1. A prosthetic heart valve, comprising:
a plurality of flexible members defining a prosthetic valve aperture adapted to open and close in response to a flow of blood produced by a heart, said plurality of flexible members including a first layer of a polymeric material, a second layer of the polymeric material, and a central porous polymeric membrane sandwiched between the first layer and the second layer.

2. The prosthetic heart valve of claim 1, wherein:
the polymeric material includes a block copolymer.

3. The prosthetic heart valve of claim 2, wherein:
the block copolymer includes polyisobutylene.

4. The prosthetic heart valve of claim 2, wherein:
the block copolymer includes block units of polystyrene-polyisobutylene-polystyrene.

5. The prosthetic heart valve of claim 4, wherein:
the central porous polymeric membrane is dip-coated into a solution of the polymeric material.

6. The prosthetic heart valve of claim 4, wherein:
the central porous polymeric membrane includes polyethylene terephthalate.

7. The prosthetic heart valve of claim 4, wherein:
the central porous polymeric membrane includes a compound selected from the group consisting of silicone rubber, polyurethane, polyolefin, nylon, expanded polyfluoroethylene, and combinations thereof.

8. The prosthetic heart valve of claim 4, wherein:
the plurality of flexible members includes three flexible members supported by a support structure, the support structure having a base and three struts that extend substantially vertically from the base.

9. The prosthetic heart valve of claim 8, wherein:
the support structure is substantially annular.

10. The prosthetic heart valve of claim 8, wherein:
the support structure includes a cuff for affixing the prosthetic heart valve to a vascular implant site.

11. The prosthetic heart valve of claim 8, wherein:
the support structure includes polyisobutylene.

12. The prosthetic heart valve of claim 1, wherein:
the prosthetic heart valve is collapsible in the radial direction.

13. The prosthetic heart valve of claim 1, further comprising:
at least one antithrombogenic agent loaded into the prosthetic heart valve.

14. The prosthetic heart valve of claim 1, further comprising:
at least one tissue growth agent loaded into the prosthetic heart valve.

15. The prosthetic heart valve of claim 1, wherein:
the central porous polymeric membrane is a polymeric fabric.

16. The prosthetic heart valve of claim 15, wherein:
said polymeric fabric includes polyethylene terephthalate.

17. The prosthetic heart valve of claim 16, wherein:
a composite multilayer polymeric membrane is formed by compression molding whereby the polymeric fabric is sandwiched between two outer polymeric layers.

18. The prosthetic heart valve of claim 17, wherein:
the compression molding partially presses the two outer polymeric layers through the polymeric fabric.

19. The prosthetic heart valve of claim 17, wherein:
the compression molding is adapted such that there is a plane of the polymeric fabric not physically integrated with the two outer polymeric layers.

20. The prosthetic heart valve of claim 1, wherein:
the plurality of members are loaded with at least one antithrombogenic agent.

21. The prosthetic heart valve of claim 1, wherein:
the plurality of members are loaded with at least one tissue growth agent.

22. The prosthetic heart valve of claim of 8, wherein:
sutures join the three flexible members to the support structure.

23. The prosthetic heart valve of claim of claim 22, wherein:
a cuff surrounds a bottom portion of the support structure, the cuff for affixing the prosthetic heart valve to a wall of a vascular implant site.

24. The prosthetic heart valve of claim of claim 23, wherein:
the cuff can be loaded with therapeutic agents.

25. A prosthetic heart valve, comprising:
a plurality of flexible members defining a prosthetic valve aperture adapted to open and close in response to a flow of blood produced by a heart, each flexible member including a polymeric composite, the polymeric composite including a polymeric fabric and two outer polymeric layers wherein the polymeric fabric is surrounded by the two outer polymeric layers, the two outer polymeric layers forming a coating on said polymeric fabric.

26. The composite multilayer polymeric material of claim 25, wherein:
the coating is made by compression molding of the polymeric fabric between the two outer polymeric layers.

27. The composite multilayer polymeric material of claim 25, wherein:
the coating is made by dip coating the polymeric fabric into a solution of a block copolymer material.

28. A method for manufacturing a prosthetic valve comprising the steps of:
a) providing a tubular polymeric structure having a top portion, a bottom portion, and defining a central axis, the top portion realized from a composite including an intermediate porous polymeric fabric disposed between two or more outer polymeric layers;
b) inserting the polymeric structure through a stent support structure, the stent support structure having a plurality of struts, the struts projecting substantially parallel to the central axis of the tubular structure;
c) forming a plurality of leaflet members from the top portion of the cylindrical polymeric structure;
d) suturing the leaflet members to the stent support structure; and
e) folding a bottom portion of the tubular polymeric structure upon itself to form an anchoring cuff.

29. The method of claim 28, wherein:
the intermediate porous polymeric fabric comprises polyethylene terephthalate and the outer polymeric layers comprise polyisobutylene.

30. The method of claim 29, wherein:
the outer polymeric layers comprise a block copolymer of polystyrene-polyisobutylene-polystyrene.

31. The method of claim 28, wherein:
the intermediate porous fabric is compression molded between the outer polymeric layers.

32. The method of claim 28, wherein:
the intermediate porous polymeric fabric is dip-coated between the outer polymeric layers.
